(19) United States
(12) Patent Application Publication
(10) Pub. No.: US 2026/0248628 A1
(43) Pub. Date: Aug. 27, 2026

(54) ENHANCED VEIN OF GALEN PROCEDURES ACCESS DEVICES AND RELATED PROCESSES

(71) Applicant: Charles Emmett Stout, Redlands, CA (US)

(72) Inventor: Charles Emmett Stout, Redlands, CA (US)

(73) Assignee: Carles t Emmett Stout, Redlands, CA (US)

(21) Appl. No.: 19/546,622

(22) Filed: Feb. 23, 2026

Related U.S. Application Data
(60) Provisional application No. 63/764,034, filed on Feb. 27, 2025.

(51) Int. Cl. A61F 2/91; A61F 2/958

(52) U.S. Cl. CPC A61F 2/91; A61F 2/958; A61F 2250/0039

(57) ABSTRACT

The Great Cerebral Vein, or Vein of Galen is comprised of a unique structure having a location which both implicates it in various clinical conditions, and can be treated by systems which, for example, go around corners of the Transverse and Straight sinuses for minimally invasive therapy embedded in the Vein of Galen the present inventions constitute progress in science and the useful arts worthy of protection as US Letters patent as novel.

### CROSS-REFERENCE TO RELATED APPLICATIONS

The present application claims priority to U.S. Provisional Application No. 63/764,034, filed Feb. 27, 2025, the contents of which are incorporated herein by reference in their entirety.

### FIELD OF THE INVENTION

The field of the invention is middle and back of the brain treatment procedures, including non-obliterative paradigms for the preservation of sinusoidal capillaries, along with improved versions of extant processes, including newly applied vessel approaches such as addressed herein, along with improved systems for the same as needed to procure desired outcomes.

Likewise, devices and systems to support these procedures are taught and disclosed in terms of structure specifically focused upon their function, especially as it relates to surgical removal of a thrombus within a subject blood vessel. Those skilled in the art understand this definitional approach to embrace any related art used during procedures having for example antegrade blood flow, wherein consequences of previously engaged emboli becoming partially or fully disengaged are dire.

### OBJECTS AND SUMMARY OF THE INVENTIONS

The present inventions comprise novel enhanced treatment systems for Vein of Galen related issues and/or improved devices having at least about a plurality of, or at least three vessel segments. By de-pressurizing select segments of the cerebral vasculature within the transverse and straight sinuas along with modulating and baffling blood flow, new approaches are taught herein along with materials and properties impacting flow dynamics linked to the solutions claimed herein and embraced within the scope of the instant application for United Staters Letters Patent as herewith first disclosed in any form or reduced to practice by this written description.

### BRIEF DESCRIPTION OF THE DRAWINGS

Various preferred embodiments are described herein with references to the drawings in which merely illustrative views are offered for consideration, whereby:

FIG. 1 shows an image of the back of the brain with prominent vessels and underserved regions according to current thinking; and,

FIG. 2 likewise shows a detailed view of an access path to the Vein of Galen particularly the Jugular veins, Transverse Sinuses and the straight sinus.

Artisans understand the relational bases of the scope based upon the teachings herein, it is respectfully proposed. Pre-existing state of the art, for example in U.S. Pat. No. 11,931,485 shows that current approaches teach away from this vessel-sparing paradigm. Likewise, using the alternative to this idea, namely novel improved Resorbable and/or eluting means along with protein capture and deposition (for example sufloxybetaines et seq.) and all image enhancement and visualization are likewise contemplated as within the scope of the instant teachings, as back-up therapy.

Corresponding reference characters indicate corresponding components throughout the several views of the drawings. Skilled artisans will appreciate that elements in the figures are illustrated for simplicity and clarity and have not necessarily been drawn to scale. For example, the dimensions of some of the elements in the figures may be exaggerated relative to other elements to help to improve understanding of various embodiments of the present invention. Also, common but well-understood elements that are useful or necessary in a commercially feasible embodiment are often not depicted in order to facilitate a less obstructed view of these various embodiments of the present invention.

### DETAILED DESCRIPTION OF THE INVENTIONS

The present inventor has made improvement over the standard of care in the Vein of Galen procedures for transvenous embolization and intracranialn venpous sinus stenting.

The Great Cerebral Vein, or Vein of Galen (used interchangeably) is comprised of a unique structure having a location which both implicates it in various clinical conditions, and begs the question of why specialized devices, for example partially open-celled stenting units, have not been emplaced therein on a routine basis to date to treat lesions and address related pathologies—including a lack of drainage. By describing and designing systems which, for example, go around corners of the Transverse and Straight sinuses for minimally invasive therapy embedded in the Vein of Galen the present inventions constitute progress in science and the useful arts worthy of protection as US Letters patent.

The description includes information that may be useful in understanding the present invention. It is not an admission that any of the information provided herein is prior art or relevant to the presently claimed invention, or that any publication specifically or implicitly referenced is prior art.

As claimed below, solutions to the problem that during thrombectomy with antegrade blood flow, engaged emboli can become dislodged from the mechanical device. Blood flow has a water-hammer effect on the emboli which can break pieces of the emboli off or entirely dislodge the emboli from the capturing device. Avoiding this until now dangerous progression is among the objects of the instant teachings, it is respectfully submitted.

In most cases, the dislodged emboli or fragments thereof can either re-occlude the original vessel or occlude a new vessel or territory.

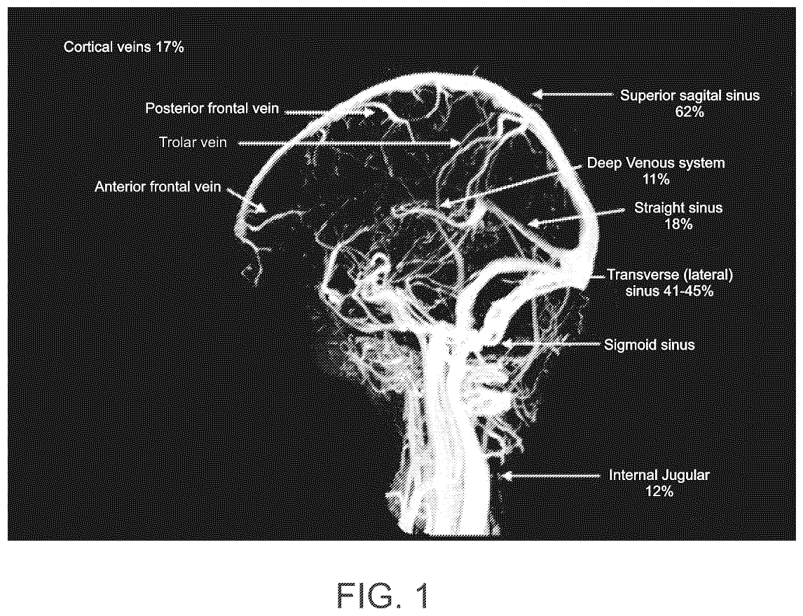

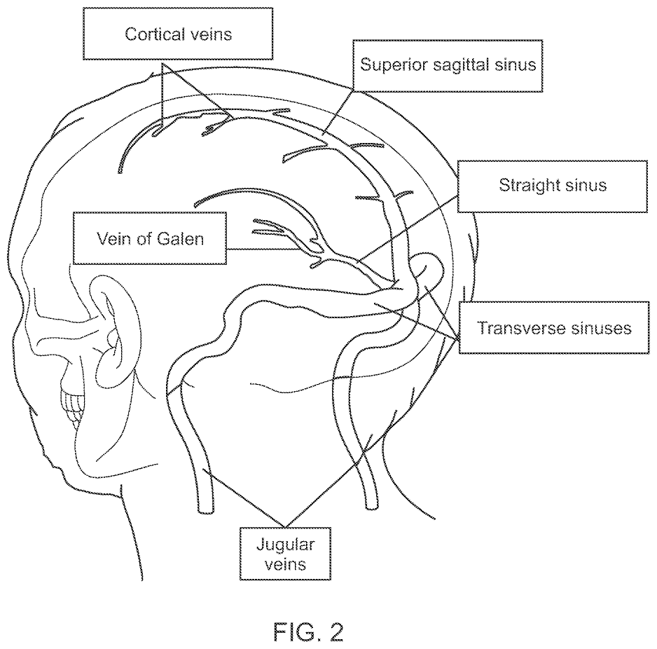

Referring now to FIG. 1, FIG. 2 the access through the Jugular veins is shown around the corner from transverse to straight sinuses.

The present inventor has shown that, using a simple 3 segment stenting-member a drop in pressure and pulsatility often allows for healing of the sinusoidal capillaries and resultory subdural hematomas. This option to vessel sacrificing may support improved treatment of many patients.

By way of further exemplary embodiments, the present inventive subject matter is drawn to compositions, devices, and methods of thrombectomy-like devices that have a mechanical self expanding device with 2 or more than 2 diameters. Referring both to FIG. 1-3, such stenting members may also work in other areas—for example one is sized appropriately for the targeted occluded vessel of 3-6 mm, and a second diameter proximal to the main diameter that is sized for vessel diameters of the internal carotid, common carotid Basilar or vertebral arteries of 9-10 mm. Having a larger proximal diameter will act to baffle the rate of blood flow.

In addition the larger baffle can be covered with a material such as PTFE or polyurethan film or a braid of polymer of metal to further enhance the baffle properties of the larger diameter.

The illustrative “barrel stent” is the inspiration for the idea. The bulge can be more proximal and adjacent to the guidewire junction.

Also the distal tip can be configured with closed end or porous end. The distal tip can be closed with a laser cut configuration and/or covered with PTFE, or a braid of polymer or metal.

Various objects, features, aspects and advantages of the inventive subject matter will become more apparent from the following detailed description of preferred embodiments, along with the accompanying drawing figure.

The inventors have discovered that thrombectomy devices with improved functional characteristics, and especially with improved thrombus retention can be prepared by adjusting the diameters of the device.

While several embodiments of the present disclosure have been described and illustrated herein, those of ordinary skill in the art will readily envision a variety of other means and/or structures for performing the functions and/or obtaining the results and/or one or more of the advantages described herein, and each of such variations and/or modifications is deemed to be within the scope of the present disclosure. More generally, those skilled in the art will readily appreciate that all parameters, dimensions, materials, and configurations described herein are meant to be exemplary and that the actual parameters, dimensions, materials, and/or configurations will depend upon the specific application or applications for which the teachings of the present disclosure is/are used.

Those skilled in the art will recognize, or be able to ascertain using no more than routine experimentation, many equivalents to the specific embodiments of the disclosure described herein. It is, therefore, to be understood that the foregoing embodiments are presented by way of example only and that, within the scope of the appended claims and equivalents thereto, the disclosure may be practiced otherwise than as specifically described and claimed. The present disclosure is directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the scope of the present disclosure.

All definitions, as defined and used herein, should be understood to control over dictionary definitions, definitions in documents incorporated by reference, and/or ordinary meanings of the defined terms.

The indefinite articles “a” and “an,” as used herein in the specification and in the claims, unless clearly indicated to the contrary, should be understood to mean “at least one.” The phrase “and/or,” as used herein in the specification and in the claims, should be understood to mean “either or both” of the elements so conjoined, i.e., elements that are conjunctively present in some cases and disjunctively present in other cases. Other elements may optionally be present other than the elements specifically identified by the “and/or” clause, whether related or unrelated to those elements specifically identified, unless clearly indicated to the contrary.

Reference throughout this specification to “one embodiment” or “an embodiment” means that a particular feature, structure, or characteristic described in connection with the embodiment is included in at least one embodiment. Thus, appearances of the phrases “in one embodiment”, or “in an embodiment” in various places throughout this specification are not necessarily all referring to the same embodiment. Furthermore, the particular features, structures, or characteristics may be combined in any suitable manner in one or more embodiments.

The terms and expressions which have been employed herein are used as terms of description and not of limitation, and there is no intention, in the use of such terms and expressions, of excluding any equivalents of the features shown and described (or portions thereof), and it is recognized that various modifications are possible within the scope of the claims. Accordingly, the claims are intended to cover all such equivalents.

Reference throughout this specification to “one embodiment,” “an embodiment,” or similar language means that a particular feature, structure, or characteristic described in connection with the embodiment is included in at least one embodiment of the present invention. Thus, appearances of the phrases “in one embodiment,” “in an embodiment,” and similar throughout this specification may, but do not necessarily, all refer to the same embodiment.

Furthermore, the described features, structures, or characteristics of the invention may be combined in any suitable manner in one or more embodiments. In the following description, numerous specific details are provided to provide a thorough understanding of embodiments of the invention. One skilled in the relevant art will recognize, however, that the invention may be practiced without one or more of the specific details, or with other methods, components, materials, and so forth. In other instances, well-known structures, materials, and so forth. In other instances, well-known structures, materials, or operations are not shown or described in detail to avoid obscuring aspects of the invention.

Unless otherwise indicated, all numbers expressing quantities of ingredients, properties such as molecular weight, reaction conditions, and so forth used in the specification and claims are to be understood as being modified in all instances by the term “about.” Accordingly, unless indicated to the contrary, the numerical parameters set forth in the specification and attached claims are approximations that may vary depending upon the desired properties sought to be obtained by the present invention. At the very least, and not as an attempt to limit the application of the doctrine of equivalents to the scope of the claims, each numerical parameter should at least be construed in light of the number of reported significant digits and by applying ordinary rounding techniques. Notwithstanding that the numerical ranges and parameters setting forth the broad scope of the invention are approximations, the numerical values set forth in the specific examples are reported as precisely as possible. Any numerical value, however, inherently contains certain errors necessarily resulting from the standard deviation found in their respective testing measurements.

The terms “a,” “an,” “the” and similar referents used in the context of describing the invention (especially in the context of the following claims) are to be construed to cover both the singular and the plural, unless otherwise indicated herein or clearly contradicted by context. Recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein is intended merely to better illuminate the invention and does not pose a limitation on the scope of the invention otherwise claimed. No language in the specification should be construed as indicating any non-claimed element essential to the practice of the invention.

Groupings of alternative elements or embodiments of the invention disclosed herein are not to be construed as limitations. Each group member may be referred to and claimed individually or in any combination with other members of the group or other elements found herein. It is anticipated that one or more members of a group may be included in, or deleted from, a group for reasons of convenience and/or patentability. When any such inclusion or deletion occurs, the specification is deemed to contain the group as modified thus fulfilling the written description of all groups used in the appended claims.

Certain embodiments of this invention are described herein, including the best mode known to the inventors for carrying out the invention. Of course, variations on these described embodiments will become apparent to those of ordinary skill in the art upon reading the foregoing description. The inventor expects skilled artisans to employ such variations as appropriate, and the inventors intend for the invention to be practiced otherwise than specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

In closing, it is to be understood that the embodiments of the invention disclosed herein are illustrative of the principles of the present invention. Other modifications that may be employed are within the scope of the invention. Thus, by way of example, but not of limitation, alternative configurations of the present invention may be utilized in accordance with the teachings herein. Accordingly, the present invention is not limited to that precisely as shown and described.

What is claimed is:

1. An enhanced VEIN OF GALEN access and therapy delivery system, being comprised of a flexible blood flow de-pressurizing stent, used to navigate orthogonal corners from the Transverse to the Straight Sinus for restoring flow within the target zone of the Vein of Galen.

2. The system of claim 1, emplacement being driven by the catheter and wire path guiding the ability to navigate the flexible blood flow de-pressurizing stent through orthogonal corners from the Transverse to the Straight Sinus.

3. The system of claim 2, further comprising an open-celled stent.

4. The system of claim 3, being comprised of a re-sheathable stent.

5. The system of claim 4, wherein the vessel is the Vein of Galen, and the stenting means allows heavy stenosis to be overcome allowing for flow through said Vein of Galen/Great Cerebral Vein.

6. The system of claim 5, being endovascularly delivered, and allowing for embolization without sacking or sacrificing of any vessels including sinusoidal capillaries by means of pressure modulation imparted by the stenting members configuration of tapers.

7. The system of claim 6, the stenting-member having at least more than one stent section or a zone of different diameter.

8. The system of claim 7, whereby a distal taper section further modulates the pressure permitting the vessel to heal in the absence of pressure.

9. A kit comprising, in combination:
access tools and for the system of claims 8; whereby the stenotic pressure is mitigated, extenuated or prevented thereby making the likelihood of blockage minimized and allowing flow through the Vein of Galen.

10. The kit of claim 9, further comprised of an open celled stent, which is re-sheathable.

11. A method of treatment for Vein of Galen insults or injuries, comprised of, in combination:
Access through the Jugular veins;
checking vessel size of the trunk of the Transverse and Straight sinuses;
controlling the flow of proximal trunk to match the size of the common trunk;
further modulating pressure with variegated distal segments; and
addressing the lesion to allow drainage from the deep brain.

12. The method of claim 11, whereby decreased pressure allows for congestion relief and decompression by the emplacement of a stenting member comprised of de-pressurizing stent segments, being open-celled and tapered drainage to the venous side.

13. The method of claim 12, whereby at least one other therapy is delivered from the group of anti-platelet medicaments; liquid embolics; embolization coils; resorbable and non-resorbable plugs.

14. The method of claim 13, whereby vessel pre-dilation is done with a micro-balloon;
a wire is emplaced;
balloon emplacement along the wire enables stent to corner with appropriate radial force; and
emplacement is managed with visualization for at least one of an open-cell, hybrid cell, tapered woven stents and laser generated stenting assembly.

15. The method of claim 14, further comprised of those secondary therapies clinically indicated.

16. The method of claim 15, which may be repeated, as needed.

17. Stents which may be re-sheathed, related to the ratio of Vein of Galen trunk size to Straight sinus and Transverse sinus dimensions of the patient's anatomy, comprised of:
pressure-relief means for sparing vessels allowing for the treatment and healing of sinusoidal capillaries;
being delivered in an out-patient setting;
being poly-sectioned stenting assemblies.

18. The stents of claim 17, delivered as primary therapy within Vein of Galen.

19. The stents of claim 18, using primary, secondary, tertiary therapies as needed, whereby drainage is enabled through the Vein of Galen.

20. The stents of claim 19, being at least partially open-celled, and re-sheathable used for at least one of transvenous embolization and intracranial venous sinus stenting with results equal to or better than the state of the art measured by the CAROTID WALLSTENT and ATLAS systems.
